(19) United States
(12) Patent Application Publication
(10) Pub. No.: US 2010/0121157 A1
(43) Pub. Date: May 13, 2010

(54) WIRELESS SENSOR RESIDENT ANNOTATIONS

(75) Inventors: Javier Espina, Aachen (DE); Martin Elixmann, Aachen (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/526,993

(22) PCT Filed: Jan. 25, 2008
(86) PCT No.: PCT/IB08/50282

§ 371 Date: Aug. 13, 2009

Related U.S. Application Data
(60) Provisional application No. 60/889,954, filed on Feb. 15, 2007.

(51) Int. Cl. A61B 5/00; G06F 17/30; G08B 1/08; G06F 17/00

(52) U.S. Cl. 600/301; 707/802; 340/539.1; 715/230; 707/E17.044

(57) ABSTRACT

A high percentage of preventable medical errors occur due to poor communication during the period when patient care is handed from one caregiver to another. Electronic annotations from patient specific body sensor networks are created and classified according to the caregiver and the urgency of the annotation to ensure that no information goes unnoticed during the transfer of care. Sensors (10) of each body sensor network store the annotations, and share the annotations with all the other sensors in the network. The sensors (10) communicate wirelessly with a monitor (12), nurses station (14) a portable device (16) or other device (19) associated with a medical facility network to display the annotations for the caregiver on an associated display (12a, 14a, 16a, 19a). The caregiver can view, create, edit, and delete annotations with input devices (12b, 14b, 16b, 19b) as appropriate. The system can be applied in a hospital or care facility in which patients are monitored with a body sensor network based monitoring system. This includes ICUs, ERs ORs, preparation rooms, cathlabs, and diagnostic imaging rooms, as well as others.

The present application relates to patient care. It finds particular application in the transfer of patient responsibility, or hand-off between care givers, but it can also be used in a preclinical or research monitoring setting. More specifically, it relates to reducing medical care errors by improving communication when patient care responsibility is transferred or modified, and will be described with particular attention thereto. It is to be appreciated, however, that the present application is also applicable to any long term care situation, and not only in a hospital patient hand-off setting.

The transfer of patient data and care is commonly referred to as “hand-off.” The same healthcare provider does not remain in a healthcare environment all the time, and some patients move between healthcare environments. Different services are provided in different areas of a hospital (e.g. diagnostic imaging, surgery, recovery, and the like). Resultantly, hand-offs are inevitable as a patient progresses through their care regimen. Further, shift changes, break periods, and the like cause changes in healthcare responsibility.

With every hand-off, there comes the potential for breakdowns in communication, which on one end of the spectrum can produce inefficiency, and the same work being done more than once, and on the other end of the spectrum can lead to medical errors. These communication breakdowns can be caused by interruption, omission of information, illegible handwriting, lost information, and the like. Studies suggest that as many as 98,000 people annually die as a result of preventable medical errors. 60% of accidental serious injuries and deaths in healthcare facilities result from breakdowns in communication.

Typically, in-hospital patient monitoring is based on wired connections to sensors that measure the vital parameters of a patient's condition. These measurements are communicated to a patient monitor to be displayed. When a patient moves from place to place, in some environments, old sensors are removed and new ones replace them. In other environments, the sensors are unplugged from a display at the old site and are plugged into a display at the new site. All the cords and wires leading from a patient's sensors can be a nuisance, decreasing the comfort of the patient and requiring creative space saving solutions when space is at a premium, such as in an operating room. In still other environments wireless sensors are used.

Standardized hand off procedures can markedly improve communication, thereby reducing preventable medical errors. One of the most recognized standardized hand-off procedures is the SBAR tool (situation, background, assessment, and recommendation). This particular tool, as well as other standardization procedures, requires the introduction of cards, paper forms, or their electronic equivalent (computers, tablet PCs, PDAs, etc.) into the hospital workflow environment, which adds to overall clutter and decreases accessibility of information. Electronic hand-off information, like its paper counterpart, can be misplaced, lost, or go unnoticed, particularly if a patient is moved.

The present application provides a new and improved patient monitoring system designed to help eliminate medical errors during patient hand-off that overcomes the above-referenced problems and others.

In accordance with one aspect, A network is provided. A user uses an input device to create, modify, or delete electronic annotations. A display selectively displays the annotations to a user. A plurality of sensors that are associated with a single patient take measurements of that patient, the sensors including a memories that store data. The network also includes wireless communication devices for wirelessly communicating created annotations, annotation modifications, and annotation deletions to the sensors for storage on at least one of the memories and for communicating annotations wirelessly to at least one of the displays.

In accordance with another aspect, a method of compiling medical annotations is provided. A plurality of sensors is connected to a subject to form a body sensor network. Annotations are entered. The annotations are wirelessly transmitted to the body sensor network for storage therein. The stored annotations from the body sensor network are wirelessly transmitted to a monitor device for display.

In accordance with another aspect, a method of handing off a patient is provided. Wireless sensor devices are placed in close proximity to a patient. The sensor devices interconnect with each other to form a body sensor network, resulting in all of the sensor devices carrying common data. The patient is moved out of a wireless communication range of a first patient monitor and into the wireless communication range of a second patient monitor, the body sensor network transmitting annotations to the second monitor after the move. The presentation displayed on the patient monitor is changed based on the identity of a healthcare professional accepting the patient hand-off.

In accordance with another aspect A body sensor network is provided. A plurality of sensors for store annotations pertaining to a subject. A device selectively displays annotations communicated from the sensors based on an annotation category.

One advantage is that the possibility for communication breakdowns during hand-off procedures is reduced.

Another advantage lies in improved versatility.

Another advantage is that it facilitates patient hand off.

Another advantage is that is applicable to a wide variety of monitoring situations.

Another advantage is that annotations remain with a patient.

Another advantage resides in application to research monitoring.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

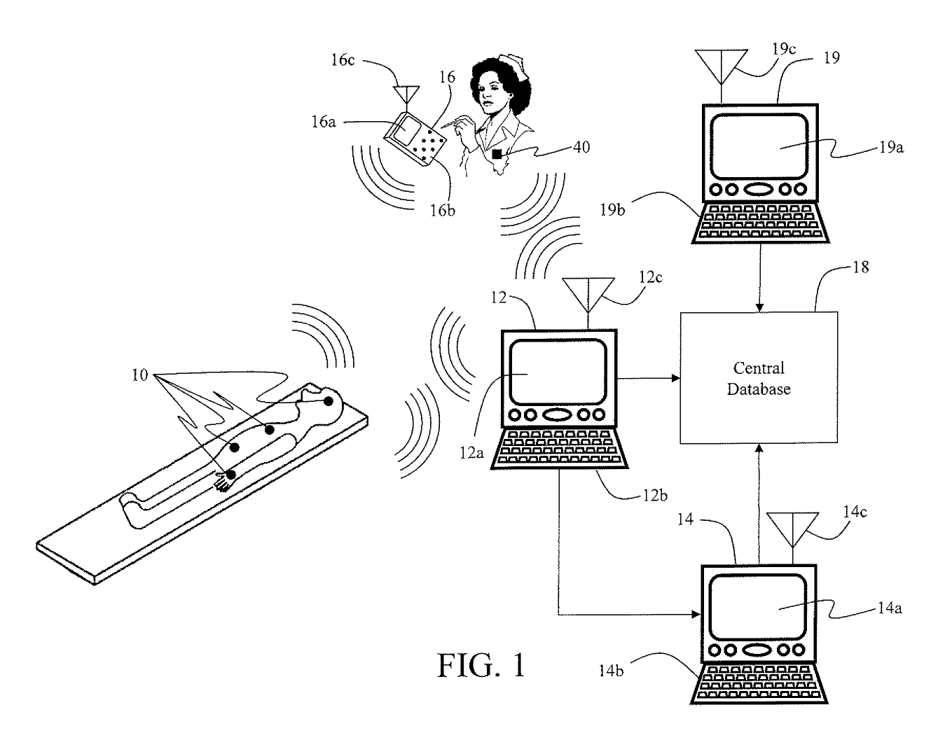

FIG. 1 is depiction of several typical components of a body sensor network;

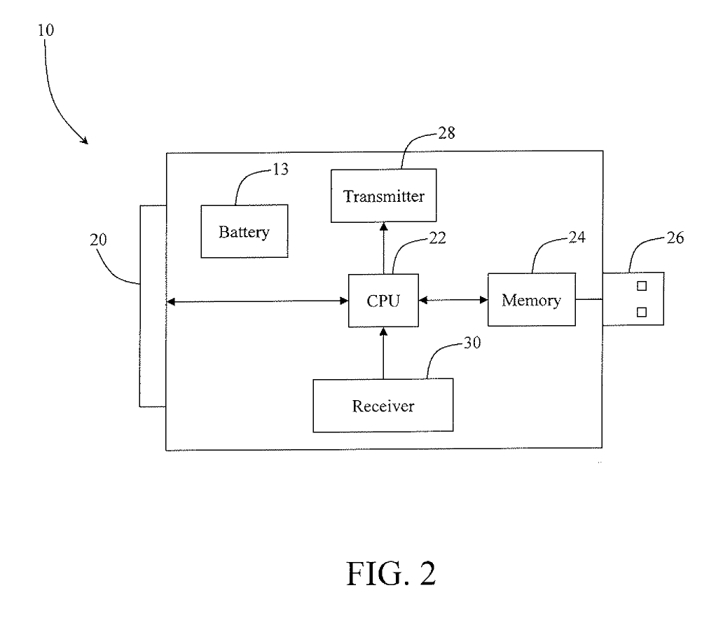

FIG. 2 is a schematic diagram of a body sensor, in accordance with the present application.

The present application contemplates the use of fully unobtrusive and flexible monitoring of all patients, from non-mobile to mobile patients, and even high acuity patients, such as intensive care unit (ICU) patients. The application also contemplates use in a non-clinical setting, a preclinical setting, and a research setting.

Through the use of Body Sensor Networks (BSNs) wireless sensors are used. By using the building blocks of a BSN, a foundation is laid to support the management of electronic annotations. With reference to FIG. 1, a medical BSN includes a collection of wireless medical sensors 10 that transmit their measurements to a network interface device 12, 14, 16, 19, such as bedside patient monitor 12 in the vicinity (within, e.g., about 5 meters of the patient), typically associated to a patient display 12a. The sensors 10 can be nodes of a body network that communicate with each other and the monitor 12 by a short range, low power wireless communication technology such as Bluetooth or ZigBee. Alternately, low power body coupled communication is contemplated for communication between the nodes of the body network, the transmissions to the bedside monitor 12 being performed by a specified node with a larger battery. Each sensor 10 has a battery 13 (shown in FIG. 2) that powers the functional elements of the sensor 10. The battery 13 is preferably light weight, and rechargeable, such as a rechargeable lithium ion cell. Other rechargeable batteries and disposable batteries are, of course, contemplated.

The bedside monitor 12 acts as a user interface device for patient related annotations and may be used by a healthcare professional to input, edit, visualize, or delete annotations. An annotation is a piece of information about a given patient. An annotation is typically information input by a physician, nurse, physical therapist, or other healthcare professional concerning a patient's situation, the patient's background, or the patient's assessment and recommendations, particularly recommendations for treatment and care. Typical annotations include medication schedules, bathing schedules, in person visit and motivational schedules, patient's medical conditions, allergies, medicine, interaction warnings, etc. The annotation can be generated anywhere, not necessarily only at the bedside monitor 12, such as from a physician's office or an electronic device 16 (PDA Tablet PC, Blackberry, etc.) that is connected with the hospital network, a nursing station, 14 or the like.

The sensors 10 periodically or continuously take measurements of a patient's vital signs and these measurements are displayed on the bedside monitor 12 and communicated to the nurses' station and a central database 18 for long term storage. For instance, a blood oxygen monitor continually monitors the patient's pulse, and blood oxygen levels. If the sensors 10 sense a medically significant or threatening event, such as a cardiac arrhythmia, the sensor 10 can automatically generate an annotation reporting the event. It is contemplated that any measurement or monitoring action taken by the sensors 10 can be presented to the healthcare professional in the form of an annotation. That is, the raw data taken by the sensors 10 can be presented as annotations. Another example of an annotation that can be created by a sensor 10 is a “battery low” annotation. If a sensor's battery is low, then it can generate an annotation requesting that the healthcare professional replace or recharge the sensor's 10 battery.

Another example of an event that could trigger annotations could come in association with on-demand pain medication, sometimes prescribed for patients' comfort after particularly rigorous procedures. In this example, when the patient presses a plunger, they receive a measured dose of pain medication. This event may trigger the automatic creation of an annotation, in response to the patient's request. A plurality of pain medication doses could be compiled into a single annotation, such as “the patient requested three doses of pain medication at 1:00 PM, 1:30 PM and 2:00 PM.” Annotations taken at the times where the patient is feeling the most pain may help the health care professionals better treat the patient's pain in the future.

Healthcare professionals associated with the patient enter their own annotations, remove old, outdated annotations, denote tasks in annotations as being completed, or the like. Again, this can be done via the bedside monitor 12, at the nurses' station 14, from other wireless devices 16, or from other workstations 19 on the hospital network. Each of the network interface devices 12, 14, 16, 19 has an associated display 12a, 14a, 16a, 19a, an interface 12b, 14b, 16b, 19b through which the healthcare professional can enter, modify, delete, etc. the annotations, and a wireless transceiver 12c, 14c, 16c, 19c. An example of an annotation entered by a healthcare professional could be a record of medications administered. Another example could be a physician's comment about the patient's mental state at a certain time. The possibilities are virtually boundless, but generally, any note that a healthcare professional would want to add to the patient's chart or file can be entered as an annotation. Typical annotations also include to do and have done lists, and other information, which will assist the next care giver to provide a continuity of care without duplications or omissions.

Having described the nature of annotations, the application now moves on to a manner of classification of the annotations. It is contemplated that the annotations will be classified on several parameters, including, but not limited to urgency of the annotation, identity of the person viewing the annotation, age of the annotation, and whether the annotation has previously been viewed.

A first possible category of annotation is “active.” An annotation is assigned the active classification if it is new, and has not been viewed by the current attending health care professional yet. When there is one or more active annotations concerning a certain patient, the network interface device 12, 14, 16, 19 can indicate this. Specifically, the display 12a, 14a, 16a, 19a can automatically display active annotations. It can display an icon indicating that there are active annotations. The network interface device 12, 14, 16, 19 can give an audible chirp if it senses a healthcare professional that has not seen the annotation yet. Sensing the identity of a healthcare professional is discussed more below. Alternately, the network interface device 12, 14, 16, 19 could utilize a combination of any of these notices, as well as others. Other active annotations include lists of activities scheduled for a next shift, such as times to give medications, daily activities, such as a patient bath, which have and have not been performed, unstable physiological parameters that need watching, and the like.

Another category of annotation is “noticed.” An active annotation is downgraded to “noticed” after a healthcare professional takes note of the annotation. The healthcare professional can change an active annotation to noticed from the bedside monitor 12, the nurses' station 14, or from any other wireless device 16, or from other input stations 19 associated with the hospital network, as noted above. In one embodiment, the “noticed” annotations are not automatically displayed to the healthcare professional after its initial viewing, but can be recalled by the healthcare professional at will, should the need arise. A healthcare professional may create an annotation and designate it as noticed, for example, when making a note for the next shift. The healthcare professional could create it as noticed and assign a time when it should become active, or it could automatically become active when the monitor 12 senses a new healthcare professional.

Another category of annotation is the “cancelled” annotation. A cancelled annotation is one that is no longer relevant, and will not be displayed to the current healthcare professional, or any later professionals. An example of a cancelled annotation might be a medication that has been dispensed, (although a record of all dispensed medications is retained in the central database 18), a task that has already been completed, and the like. A cancelled annotation is one that is not likely to be relevant in the present or future. Cancelled annotations can be periodically offloaded to the central database 18 for long term storage. From there they can be accessed at a later date should the need ever arise.

Another possible category of annotation is “critical.” These annotations indicate an emergency situation where immediate action by the healthcare professional is required. For example, a pulse sensor might sense that the patient no longer has a pulse. The sensor 10 would generate a critical annotation and possibly trigger an alarm to get a healthcare professional's attention. The sensors can work together in this regard. In another example, the pulse sensor detects no pulse, but an ECG sensor detects a normal heart rhythm. In this case the sensors know that the patient's heart has not stopped, but the BSN may yet create an active annotation requesting the attention of the current healthcare professional, such as “check pulse sensor.” Perhaps the pulse sensor has slipped off, or circulation to that area has become poor for some reason. In this example two sensors 10 work together to avoid creating a critical annotation false alarm, but they nonetheless create a lesser degree of annotation.

A more detailed depiction of an exemplary sensor 10 is shown in FIG. 2. Each sensor 10 has a sensing device 20, although the body network may have nodes that do not include a sensing device. There are a wide variety of possible sensors 10. Already mentioned were pulse and blood oxygen sensors, ECG sensors, and blood pressure sensors. Other sensors might include IV flow rate sensors, temperature sensors, brain wave sensors, and the like. A plurality of functions may further be combined into a single sensor. The sensing device 20 is connected to a sensor CPU 22, which coordinates all of the activities of the sensor 10. Typically, the CPU directs the sensing device 20 to take a measurement when appropriate, and stores the resultant measurement in an on-board memory 24. Optionally, the memory can be accessible by a physical connection 26, such as universal serial bus (USB) connection, IEEE 1394 (firewire) connection, or other similar high-speed data transfer protocol. As mentioned before, the memory 24 can be cleared of cancelled annotations every so often by downloading them to the central database 18 then deleting them from the memory 24. Typically, the sensors 10 operate independently and create annotations whenever appropriate or requested.

For its wireless communication purposes, the sensor 10 includes a wireless transmitter 28, e.g. a Bluetooth or a ZigBee transmitter. The sensor 10 uses the wireless transmitter 28 during normal operation, to send its sensor measurements to other sensors or nodes in the network, to the bedside monitor 12, nurses' station 14, or other device 16. Sometimes, such as when a patient is in transport from one area of a hospital to another, a monitor 12 is not within range of the sensors 10. If this is the case, the sensors continue to store measurements and annotations, if any, and send them as soon as a hospital network interface device 12, 14, 16, 19 is in range.

The sensor 10 also includes a wireless receiver 30, e.g. a Bluetooth or a ZigBee receiver to receive communications from other sensors 10 or nodes or from the bedside monitor 12. The sensors 10 can communicate with each other, other nodes, the bedside monitor 12, nurses' station 14, other receivers on the hospital network 19 and other electronic devices 16. Alternately, the sensors communicate with each other and a node by a body coupled communications protocol. The node, such as a wrist band, which carries the body coupled communications transmitter and receiver described above and also a wireless, e.g. Bluetooth or ZigBee transmitter and receiver for communicating with the base station associated with the bedside monitor 12 or other hospital network transmitters and receivers 19, or medical professional communication devices 16. The node can also carry a larger battery pack than the other sensors 10 to power the wireless transmissions. Optionally, the node carries the patient ID software to coordinate the adding or removing of sensors from the body network, encryption and decryption software, and the like.

Annotations from medical professionals are transmitted wirelessly to the BSN, either through a designated node or to each sensor individually. The sensors 10 of the BSN are in communication with each other. The sensors 10 routinely send their own readings and annotations to the other sensors 10 in the BSN while receiving readings and annotations from all of the other sensors 10 in the BSN. In one embodiment, all of the sensors 10 within a BSN are updated with the most current annotations concerning the patient. This way, all of the sensors 10 carry the same annotations in their respective resident memories 24. Thus, if one sensor 10 is removed from the patient's body, malfunctions, or becomes unable to transmit in some other fashion, readings and annotations resident on that sensor's memory 24 are redundantly stored on the other sensors and nodes. This way, as long as at least one sensor 10 remains with the patient (it doesn't matter which one) measurements and annotations concerning that patient will be preserved and readily available.

Following this model, healthcare professionals can swap sensors in and out of the BSN at will, and not all sensors need to be used all the time. For example, perhaps an ultrasound sensor is placed on an expectant mother's abdomen to monitor a fetus's heart rate. Once the baby is born, the sensor is no longer needed, so it can be removed from the patient's body, but the contents of its memory 24 will safely be resident in all of the remaining sensors 10 in the BSN, even if they have not been communicated to a bedside monitor 12, nurses' station 14, or other device 16.

It is preferable that all of the sensors 10 in the BSN store all of the measurements and annotations, so as long as one sensor remains, all of the measurements and annotations remain. Alternately, a master sensor or node that collects and stores the measurements and annotations could be used.

To introduce a sensor 10 into an already existing BSN, the new sensor 10 first must be associated with that BSN. A fresh sensor 10, such as one that is new, or one that was recently recharging, will not be associated with any BSN. The healthcare professional first associates the fresh sensor with the desired BSN. Each BSN will have a unique identifier, which is then tied back to the identity of the patient. It is to be understood, however, that the identity of the patient does not need to be known. For example, if an unconscious trauma victim is brought to the emergency room by paramedics, but has no identification, the attending staff will assign the patient a unique hospital number and associate the BSN with that number. So even though the patient's identity is not known, the patient's BSN will nevertheless be associated with the patient.

All communications to or from the sensors 10 will include the BSN identifier. This is to prevent cross-talk between BSNs. For example, Patient 1 and Patient 2 each have their own BSN. If the two patients sit down at the same table to have lunch together, there will be no cross-talk between the networks. Since each BSN has its own identifier, Patient 2′s sensors will ignore communications from Patient 1′s sensors, because they are not associated with the same BSN. The sensors 10 could be actively or passively associated with each other to form the patient's BSN.

Situations may arise where an associated sensor 10 is removed from the BSN or re-associated with a different BSN. In this situation, the redundancy of the BSN keeps data from being lost. Even though the removed sensor 10 is no longer in the BSN, all data carried in its memory will have been already communicated to other sensors in the BSN. So long as there is still at least one sensor associated with the BSN, data from any removed sensors 10 will not be lost. Further, every annotation carries a unique identifier, which identifies, among other things, the BSN (patient) with which the annotation is associated when the annotation was made.

To avoid cluttered communications with the patient monitor 12, one sensor 10 in the BSN can assume a temporary lead role. For example, if a patient has ten sensors 10 associated with their BSN, it may be undesirable to have all ten sensors 10 trying to communicate with a patient monitor 12. One of the sensors 10, perhaps the sensor 10 that has been associated for the longest period of time, could assume a temporary lead role. In this situation, all of the sensors 10 would continue to share annotations with all other sensors 10 in the BSN, but only one would communicate outside the BSN. If the temporary lead sensor 10 is removed from the BSN, no data is lost (again since the sensors 10 do not stop sharing data) but a different sensor 10 assumes the lead role. This might happen after sensing no communications from the previous lead sensor 10 for a period of time.

Once a patient's BSN is running and associated, patient hand-offs can now occur. As mentioned above, a patient hand-off can come in the form of a patient relocation (e.g. from surgery to recovery) or a shift change (e.g. nurse 2 takes over for nurse 1). In both cases, each patient monitor 12 can be associated with a different BSN, or at least be able to distinguish between different BSNs. To illustrate, say a patient is waiting for surgery in a pre-op room. Beside their bed is a monitor 12 associated with their BSN. Another patient monitor 12 awaits in the operating room. When the patient is wheeled from pre-op to the operating room, the pre-op monitor 12 no longer senses the BSN. The operating room monitor 12 senses the patient's BSN when they are wheeled in the room and immediately displays all relevant annotations. Annotations that were classified as noticed can be upgraded to active, because it is assumed that a new healthcare professional or professionals have taken over care of the patient, and those annotations may be relevant and unknown to the new professional(s). Of course, new annotations that were created in the pre-op room or the operating room are also displayed on the operating room monitor 12.

If it is desirable to have uninterrupted monitoring during patient transport, a BSN enabled measurement server (MMS) can monitor measurements from the wireless sensors in the absence of a patient monitor 12. For example, the MMS can be detached from a monitor 12 in pre-op and re-attached to an operating room monitor 12. This would provide uninterrupted monitoring as the patient is in transit.

Another type of hand-off occurs with a shift change, where the patient is not moving, but a new healthcare professional assumes the patient's care. In this case, the new healthcare professional identifies themselves to the patient monitor 12 (or nurses' station 14 or other device 16 and the monitor 12 upgrades noticed annotations to active, to make sure that the new healthcare professional takes note of them. A concept that can assist in this arena is the concept of Active Digital Aura (ADA) which might be based on body coupled communications. The healthcare professional carries with them a wireless ADA transmitter 40 that identifies themselves. This allows the BSNs to automatically detect the identity of the healthcare professional, and allows the BSNs to classify annotations differently for different professionals. Otherwise, the professional would identify themselves, and manually bring up the annotations, such as by pressing a “show all annotations” button. To illustrate, Nurse 1 creates an annotation in the form of a memo that says that Patient has already had the maximum amount of pain medication allowed for the day. The memo is classified as a noticed annotation. When Nurse 1's shift ends, she forgets to tell Nurse 2 that Patient cannot have more pain medication today. But as soon as Nurse 2 walks into Patient's room or comes into close proximity of the patient or the bedside monitor 12, Nurse 2's ADA is sensed and upgrades the noticed memo to active, since Nurse 2 has not seen it yet. Nurse 2 reads the memo and the information is conveyed even though Nurse 1 forgot to mention it. The concept of Active Digital Aura ensures that as much data as possible is conveyed during a patient hand-off to help prevent medical mistakes.

The nurse, physician, or other healthcare professional can enter annotations on their PDA tablet computer, or other electronic device 16 and wirelessly communicate the annotations to the BSN for storage. Any or all of these devices may be equipped with voice recognition software to record spoken annotations. Other healthcare professionals visiting the patient can read the annotations on their electronic device 16. Note that these storage and retrieval steps are performed even in the absence of a hospital network. With a hospital network, annotations can be generated at remote locations, such as a healthcare professional's office, communicated over the hospital network to the bedside monitor 12 and wirelessly transmitted to the BSN for storage one or more sensors or nodes. The stored annotations can be retrieved, viewed, modified, and the like using the same path. When using the active digital aura or other healthcare professional identifier, the state (active, noticed, etc.) of each annotation can be differently displayed to each of a plurality of healthcare professionals that are concurrently viewing the same patient's list of annotations in the language of their choice on their viewing device.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A network comprising:
an input device usable to create, modify, or delete electronic annotations;
one or more displays that selectively displays the annotations to a user;
a plurality of sensors that are associated with a single patient and take measurements of that patient, the sensors including a memory that stores data; and,
wireless communication devices for wirelessly communicating created annotations, annotation modifications, and annotation deletions to the sensors for storage on at least one of the memories and for communicating annotations wirelessly to at least one of the displays.

2. The network as set forth in claim 1, wherein the display displays a state of the displayed annotations to the healthcare professional based on the identity of the healthcare professional.

3. The network as set forth in claim 2, further including:
an active digital aura device that identifies a healthcare professional to the network.

4. The network as set forth in claim 1, wherein the display displays a categorized annotation as one of active, noticed, and cancelled.

5. The network as set forth in claim 1, further including a central database to which annotations are uploaded for long-term storage.

6. The network as set forth in claim 1, wherein the display displays an annotation that has been classified as critical, and an alarm is triggered.

7. The network as set forth in claim 1, further including:
a body sensor network including a plurality of nodes, the plurality of sensors being at least some of the nodes.

8. The network as set forth in claim 7, wherein the input device and the display are associated with a portable electronic device, which wirelessly communicates with one or more of the nodes of the body sensor network.

9. The network as set forth in claim 7, wherein the input device and the display device are associated with a patient monitor.

10. The network as set forth in claim 7, further including:
a medical facility network that includes at least one of the communications devices for communicating wirelessly with the body sensor network, at least one of the input devices, at least one display, and a central database.

11. A method of compiling medical annotations comprising:
receiving annotations pertaining to a subject;
wirelessly transmitting the annotations to the body sensor network for storage therein;
wirelessly transmitting the stored annotations from a body sensor network that includes a plurality of sensors to a patient monitor for display.

12. The method as set forth in claim 11, further including:
the sensors synchronizing the data they store so that all of the sensors in the plurality store the same data.

13. The method as set forth in claim 12, further including:
connecting an additional sensor to the subject; and
synchronizing the additional sensor with all other sensors in the subject's body sensor network.

14. The method as set forth in claim 11, further including:
wirelessly transmitting annotations from the body sensor network to a portable electronic device for display.

15. The method as set forth in claim 11, further including:
receiving annotations from a patient monitor that were created at the patient monitor.

16. The method as set forth in claim 11, further including designating each annotation as one of active, noticed, and cancelled.

17. The method as set forth in claim 11, further including:
identifying a healthcare professional viewing an annotation; and,
changing a designation of an annotation depending on the identity of the healthcare professional viewing the annotation.

18. The method as set forth in claim 17, wherein the step of identifying is performed based on a digital aura signal.

19. The method as set forth in claim 10, further including:
designating an annotation as critical and triggering an alarm.

20. The method as set forth in claim 10, further including:
wirelessly transmitting the annotations from the body sensor network to another network.

21. A body sensor network comprising:
a plurality of sensors for storing annotations pertaining to a subject;
a device for selectively displaying annotations communicated from the sensors based on an annotation category.

22. The body sensor network as set forth in claim 21, wherein the annotation category is based at least in part on an identification of a healthcare professional.

23. The body sensor network as set forth in claim 21, wherein the plurality of sensors store common data.

24. A method of handing off a patient comprising:
placing a plurality of wireless sensor devices in close proximity to a patient;
allowing the sensor devices to interconnect with each other into a body sensor network, resulting in all of the sensor devices carrying common data;
moving the patient out of a wireless communication range of a device and into the wireless communication range of a second device, the body sensor network transmitting annotations to the second device after the move;
changing a presentation displayed on a display of the device based on the identity of a healthcare professional accepting the patient hand-off.
